(19) United States
(12) Patent Application Publication
(10) Pub. No.: US 2008/0009868 A1
(43) Pub. Date: Jan. 10, 2008

(54) Device and method for treating compression fractures

(76) Inventors: Bruce Gotfried, Kiryat Bialik (IL); Yechiel Gotfried, Kiryat Motzkin (IL)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/825,639

(22) Filed: Jul. 5, 2007

Related U.S. Application Data
(60) Provisional application No. 60/806,735, filed on Jul. 7, 2006.

(51) Int. Cl. A61B 17/58; A61B 17/56

(52) U.S. Cl. 606/63; 606/72; 606/90

(57) ABSTRACT

Apparatus for treating a bone with a compression fracture, includes a coil having a compressed state and an expanded state and which is insertable into the bone when in its compressed state, and an expansion tool arranged to be coupled to the coil when the coil is present in the bone. Manual control of the expansion tool, e.g., by a physician, enables expansion of the coil from the compressed state to the expanded state. Methods for using the apparatus are also described.

### CROSS REFERENCE TO RELATED APPLICATION

This application claims priority of U.S. provisional patent application Ser. No. 60/806,735 filed Jul. 7, 2006, incorporated by reference herein.

### FIELD OF THE INVENTION

The present invention relates generally to implantable medical devices, and specifically to methods and devices for treatment of compressive bone fracture or diseased bone, such as a fractured vertebra.

### BACKGROUND OF THE INVENTION

The spinal column serves as the support structure for the body, giving the body its posture. Yet age, disease, and trauma can cause structural failures of the spinal column arising from, for example, vertebral fractures, disc hernias, and degenerative disc diseases. Such pathologies may result in pain, spinal instability, and paralysis.

The spinal column includes 26 vertebrae. A typical vertebra consists primarily of two parts—an anterior segment comprising the body of the vertebra, and a posterior segment comprising the vertebral or neural arch. The outer layer of the vertebra is made of cortex bone, and the inner portion of the vertebra is made of cancellous bone.

Typical vertebral column disorders include traumatic damages such as compression fractures, degenerative disc disease, disc hernias, scoliosis, kyphosis, and lordosis. In many instances, current treatments of vertebral compression fractures are not fully successful with respect to fractures of cancellous bone. Additionally, fractures in the thoracic and lumbar spine are common, particularly in elderly patients who suffer from weak, osteoporotic bones. Known treatments for many of these fractures are not fully satisfactory. For example, some treatments include injection of liquid bone cement into the fracture (vertebroplasty), and insertion of a prosthetic balloon that is inflated to create a cavity where cement can be subsequently injected (kyphoplasty). Other treatments include the use of expandable implants that are placed within the vertebra in an attempt to repair cancellous bone fractures of the vertebra.

U.S. Patent Application Publication No. 2003/0171812 to Grundberg et al., incorporated by reference herein, describes a modular support implant device and method that includes a plate for use in conjunction with one or more additional plates in a modular reconstructing and supporting assembly for reconstructing and supporting a diseased or fractured bone or within a space previously occupied by a diseased intervertebral disc of a patient. The plate has a small enough size to be suitable for separate insertion into the bone or the space, preferably through a cannula, and to be arranged adjacent and in combination with the other plates, for example one on top of the other to form a scaffold, so as to provide a supporting prosthesis. In another embodiment, the plate has at least two substantially opposite aspects with interlocking features designed to facilitate interlocking of adjacent plates in order to prevent or restrain the plates from sliding relative to one another.

U.S. Patent Application Publication No. 2005/0182414 to Manzi et al., incorporated by reference herein, describes a system and method for distracting opposite surfaces from the interior of a bone, such as a vertebral body. A working channel cannula provides a working channel through which an inserter and an injection cannula can simultaneously pass. The inserter transports a plurality of wafers into the interior of the bone to form a load-bearing stack bearing against the opposite surfaces. The injection cannula is used to inject a fluent material into and/or around the stack. In certain embodiments, the fluent material is a load-bearing or hardenable material, such as bone cement. In other embodiments, the fluent material can be a BMP, HAP, or other osteo-inductive, osteo-conductive, or pharmaceutical composition. A syringe containing the fluent material is engaged to the injection cannula and is operable to inject the fluent material into the vertebral body under controlled pressure.

PCT Patent Publication No. WO 03/039328 to Forester, incorporated by reference herein, describes expandable implants for intervertebral disc repair and methods and apparatus for delivering the same into the disc. The implants are described as also possibly being used for repair of bone fractures. The implants generally comprise a compressed form having a size adapted for insertion into a defect in the intervertebral disc, and a composition that allows the implant to expand from the compressed form into an expanded form after the implant is inserted into the defect. The expanded form of the implant has a configuration that fills the defect in the disc. The defect in the disc can be an annular defect that resulted from repair of a herniation of the disc, or a nucleus that needs to be repaired. The composition used to make the implant can comprise a shape memory alloy (SMA) or any other suitable material.

PCT Patent Publication No. WO 04/034924 to Grunberg et al., incorporated by reference herein, describes a minimally invasive method and device for reconstructing and supporting a fractured or diseased bone, preferably a fractured or diseased vertebra. In addition, supporting means are described for a space previously occupied by a diseased intervertebral disc, which has been completely or partially removed. The minimally invasive reconstructing and supporting spiral device comprises a coiled structure made of a coiled strap or, for example, shape-memory material, whereby the strap is advanced towards a predetermined position in a deployed state and regains a coiled shape within the target position providing a supporting prosthesis.

PCT Patent Publication WO 04/086934 to Shezifi et al., incorporated by reference herein, describes a device for distracting and supporting two substantially opposing tissue surfaces in a patient's body, to be introduced within the tissue surfaces in a minimally invasive procedure. The device comprises a wrapping element and an expandable structure insertable between two substantially opposing support surfaces of the wrapping element and adapted to be expanded between the two substantially opposing surfaces to a predetermined dimension.

U.S. Patent Application Publication No. 2005/0038517 to Carrison et al., incorporated by reference herein, describes devices, kits, and methods for treating a bone structure, e.g., a vertebra, that has sustained a compression fracture. Wedges are introduced into the bone structure in a direction that is lateral to the compression fracture, and stacked to apply forces to the bone structure to reduce the compression fracture. The wedges can be introduced into the bone structure using a cannula. The wedges can be introduced as wedge pairs, in which case, a subsequent wedge pair can be introduced between a previously introduced wedge pair in order to drive the previously introduced wedges apart to create the stacking arrangement. Optionally, the wedges can be provided with longitudinal bores, in which case, they can be introduced into the bone structure over a guide member that is threaded through the bores.

U.S. Patent Application Publication No. 2005/0209595 to Karmon, incorporated by reference herein, describes expandable devices and methods for treating and enlarging a tissue, an organ or a cavity. The device includes a hollow expanding pouch made of a resorbable material or a perforated material that can be attached to a filling element. The pouch can be filled with a biocompatible material, one or more times every few days after the insertion of the device. While filling the pouch every few days, the tissue expands and the filling material, if it is bioactive, starts to function. The devices allow immediate direct contact between the filling material and the tissue. These devices and methods are described as being able to be used, for example, for horizontal and vertical jawbone augmentation, soft tissue augmentation, and fixating bone fractures.

U.S. Pat. No. 6,981,981 to Reiley et al., incorporated by reference herein, describes systems for treating a bone, e.g., a vertebral body, having an interior volume occupied, at least in part, by cancellous bone. The systems utilize a first tool, a second tool, and a third tool. The first tool establishes a percutaneous access path to bone. The second tool is sized and configured to be introduced through the percutaneous access path to form a void that occupies less than the interior volume. The third tool places a volume of filling material within the void through the percutaneous access path. Related methods for treating a bone, e.g., a vertebral body, having an interior volume occupied at least in part by cancellous bone entail establishing a percutaneous access path to bone. A tool is introduced through the percutaneous access path and manipulated to form a void that occupies less than the interior volume. A volume of filling material is then placed within the void through the percutaneous access path. Embodiments are also described with respect to the tibia.

### SUMMARY OF THE INVENTION

In at least one embodiment of the present invention, an implantable apparatus for treating a vertebral compression fracture comprises an implant that is adapted to be placed in a compressed state within a vertebral body. It is noted that while some embodiments of the present invention are described by way of example with respect to treating a vertebral body, the scope of the present invention includes using the same or similar apparatus and methods to treat compression fractures of other bones, or to treat diseased bones that have become compressed. While the implant is within the vertebral body and still in the compressed state, it can typically be manipulated by a physician, or other person, in order to place it in a desired orientation. An expansion tool may be removably coupled to the implant and manually operated by the physician to expand the implant until it reaches a desired final state. Although the implant may have some natural springiness, resiliency or shape memory which expands it to some extent after placement within the vertebral body, the actual final level of expansion is dependent on the physician. In other words, the implant has no predetermined final deployed state. As the physician operates the expansion tool (e.g., by manually rotating the expansion tool or sliding one portion of the expansion tool with respect to another portion of the expansion tool), the implant is brought towards the final state, which is typically determined by the physician during the implantation procedure. A concept described in this paragraph and elsewhere in the specification is expanding the implant “under physician control” (“under manual control” is also used herein and has a similar definition), i.e., whereby the physician uses a tool to control the expansion and final disposition of the implant. The implant in its expanded state remains within the vertebral body following the procedure and is able to support the vertebral body, at least in part, in or near its pre-fracture shape.

Thus, for some applications, the same implant can typically be used under a variety of conditions, for example, in order to treat wedge, crush or biconcave fractures in a variety of anatomical locations, or can be placed in vertebrae of varying sizes and pathological conditions. In most if not all of these cases, the physician determines the extent of expansion of the implant.

Typically, the implant is non-inflating, and instead comprises a solid object which is unfolded or unrolled as part of its expansion. For example, the implant may comprise a sheet of metal (or another strong, flexible material) that in its compressed state is coiled like a scroll. The sheet of material may have flat or planar major sides. In one embodiment, the implant comprises a chrome-based stainless steel, such as 316 stainless steel, cobalt chrome, or a titanium-based alloy (e.g., nitinol). For some applications, the implant is thermally treated and/or has one or a plurality of holes passing therethrough that make the implant more flexible and thereby allow it to be easily manipulated. In any case, the expansion tool may typically be coupled to an inside edge region of the coiled sheet of metal and rotated in order to apply a force to the inside edge of the implant that causes the implant to expand, i.e., to increase its outer radius to a final radius deemed suitable by the physician to support the vertebral body in which it is implanted.

The expansion tool typically comprises a rigid or somewhat flexible rod that is rotated in order to cause the implant to expand.

If it is desired to reposition or remove the implant following the expansion thereof, the tool can typically, but not necessarily, be operated in a reverse direction in order to compress the implant and facilitate the repositioning or removal of the implant. For some applications, removal of the implant is performed in a follow-up procedure, by re-coupling the expansion tool to the implant, or coupling a different tool to the implant, and reducing the size of the implant using the tool.

For some applications, an autogenic or allogenic bone graft may be placed within the vertebral body after the implant has expanded to its final state. Typically, the bone graft helps to support the cylindrical shape of the implant when the vertebra is in full weight-bearing mode (e.g., when the patient is standing). In one embodiment, the sheet of metal is shaped to define a large number of holes, and bone graft placed in the implant is in enhanced communication, via the holes, with vertebral cancellous bone tissue. As appropriate, these embodiments of the present invention are practiced using techniques described in Campbell's Operative Orthopaedics, Volume One, Part I, General Principles, Chapter 2: Surgical Techniques and Approaches, Bone Grafting (Canale and Campbell, eds., Mosby, 2002), incorporated by reference herein.

There is therefore provided, in accordance with one embodiment of the invention, apparatus for treating a bone with a compression fracture, including a coil configured to be inserted in a compressed state into the bone, and an expansion tool configured to be coupled to the coil and to expand the coil under physician control while the coil is in the bone.

In one embodiment, the bone includes a vertebral body, and the coil is configured to be inserted into the vertebral body. Alternatively, when the bone includes a tibia, the coil is configured to be inserted into the tibia.

In one embodiment, the expansion tool includes a portion configured to engage the coil and to expand the coil by rotating an inner portion of the coil.

The coil may include or define a plurality of holes configured to permit communication between bone graft within the coil and native bone outside of the coil.

An outer edge of the coil may be configured to provide enhanced adhesion to the bone. Alternatively or additionally, the outer edge of the coil includes teeth which are configured to engage bone tissue and stabilize the coil in the bone.

The coil may additionally or alternatively be configured to be (a) compressible under physician control after being expanded inside the bone, to an extent sufficient to allow repositioning of the coil within the bone, and/or (b) expandable following repositioning, using the expansion tool.

In one embodiment, the coil is configured to be expandable while in the bone to a range of possible final levels of expansion, an actual final level of expansion thereof being determinable by an extent of use of the expansion tool.

There is further provided, in accordance with an embodiment of the invention, a method for treating a bone with a compression fracture, including inserting a coil in a compressed state into the bone, and expanding the coil under physician control while the coil is in the bone.

When the bone includes a vertebral body, insertion of the coil involves inserting the coil into the vertebral body. When the bone includes a tibia, insertion of the coil involves inserting the coil into the tibia.

In one embodiment, expanding the coil includes manually rotating an inner portion of the coil while an outer edge of the coil remains generally stationary.

The coil may include or be shaped to define a plurality of holes, and the method includes applying bone graft within the coil.

Inserting the coil may additionally or alternatively include securing an outer edge of the coil to native bone. In one embodiment, the outer edge includes teeth, and securing the outer edge involves stabilizing the coil in the bone by coupling the teeth to bone tissue.

In one embodiment, the method includes compressing the coil following the step of expanding the coil, repositioning the coil, and re-expanding the coil under physician control while the coil is in the bone.

In one embodiment, the method includes compressing the coil following the step of expanding the coil, and removing the compressed coil from the bone.

Expanding the coil may involve using an expansion tool to expand the coil. Expanding the coil under physician control may involve using such an expansion tool to set a final level of expansion of the coil.

There is yet further provided, in accordance with an embodiment of the invention, a method for treating a bone with a compression fracture, including inserting a device in a compressed state into a first site of the bone, expanding the device under physician control while the device is in the bone, to reduce a first location of the fracture, compressing the device while the device is in the bone, moving the device within the bone to a second site, and expanding the device under physician control while the device is at the second site, to reduce a second location of the fracture.

In one embodiment, insertion of the device involves inserting the device during a medical procedure, and the method includes maintaining the device within the bone following the procedure.

### BRIEF DESCRIPTION OF THE DRAWINGS

The present invention will be more fully understood from the following detailed description of embodiments thereof, taken together with the drawings, in which:

FIGS. 1 and 2 are schematic illustrations of a vertebral body implant in a compressed state thereof, in accordance with an embodiment of the present invention;

FIGS. 3 and 4 are schematic illustrations of the implant of FIGS. 1 and 2, in an expanded state thereof, in accordance with an embodiment of the present invention;

FIG. 5 is a schematic illustration of an exemplary expansion tool coupled to the implant of FIGS. 1-4, in accordance with an embodiment of the present invention;

FIGS. 6A and 6B are schematic anatomical drawings showing suitable implantation approaches for treating vertebral compression fractures, in accordance with various embodiments of the present invention;

FIG. 6C is a schematic anatomical drawing showing a suitable implantation approach for treating an upper tibial compression fracture, in accordance with an embodiment of the present invention;

FIGS. 7-12 are schematic illustrations showing an implantation procedure, in accordance with an embodiment of the present invention;

FIGS. 13A, 13b, 13C and 13D are schematic illustrations of the implant of FIGS. 1-4, in accordance with respective embodiments of the present invention; and

FIGS. 14A, 14B, and 14C are schematic illustrations of implantation apparatus, in accordance with respective embodiments of the present invention.

### DETAILED DESCRIPTION OF THE INVENTION

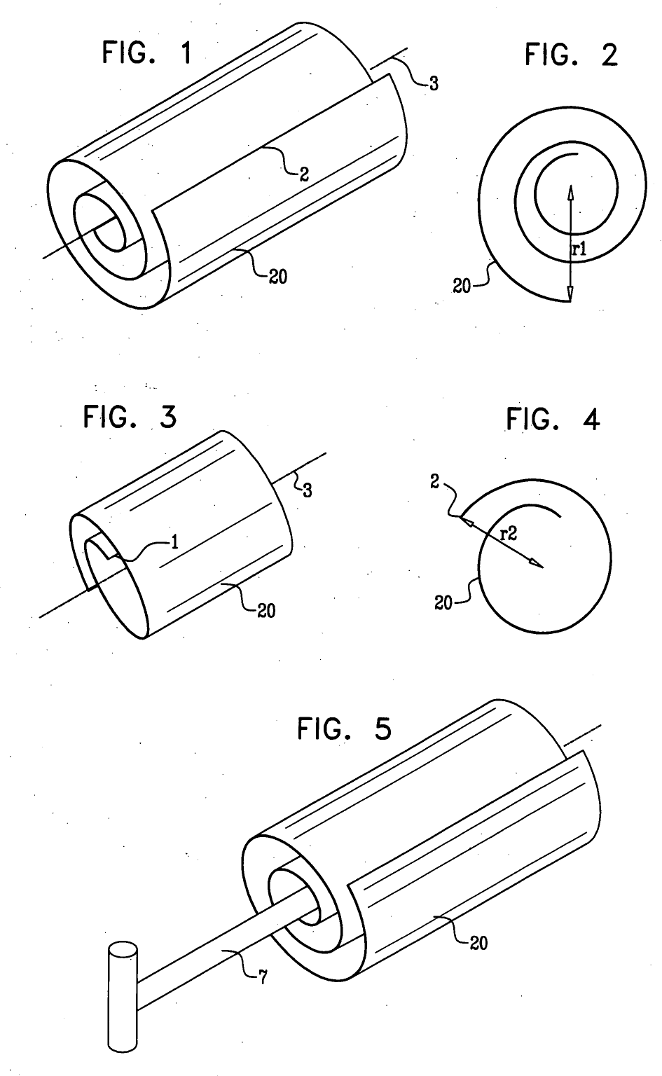

Referring to the accompanying drawings wherein like reference numerals refer to the same or similar elements, FIGS. 1 and 2 are schematic illustrations of a vertebral body implant 20 in a compressed state thereof, in accordance with an embodiment of the present invention, FIG. 2 being a cross-sectional view of the implant shown in FIG. 1. A distance r1 between an outer edge 2 and a longitudinal axis 3 of the implant is typically between about 2 mm and about 5 mm, for example about 2.5 mm. Prior to being rolled into the shape shown in FIG. 1, the dimensions of the implant are typically between about 0.1 mm and about 0.5 mm thick (e.g., about 0.2 mm), between about 10 mm and 30 mm wide (e.g., about 20 mm), and between about 50 mm and about 150 mm long (e.g., about 90 mm).

In general, the size of implant 20 is selected based on the patient's physiology. The implant 20 typically comprises a strong, flexible, biocompatible material. In one embodiment, the implant 20 comprises a chrome-based stainless steel, such as 316 stainless steel, cobalt chrome, or a titanium-based alloy (e.g., nitinol). Other materials, such as those described in the references incorporated by reference herein, are also suitable.

Referring now to FIGS. 3, 4 and 5, FIGS. 3 and 4 are schematic illustrations of the implant 20 of FIGS. 1 and 2, in an expanded state thereof, in accordance with an embodiment of the present invention, and FIG. 5 is a schematic illustration of an expansion tool 7 coupled to implant 20, in accordance with an embodiment of the present invention. The implant 20 is adapted to be placed within a vertebral body using known techniques. For example, the techniques may include (a) techniques similar to those employed in a kyphoplasty procedure in order to place a balloon within a vertebral body, (b) techniques described in one or more of the references described above, and/or (c) techniques utilizing approaches used for taking a vertebral biopsy (such as described in Campbell's Operative Orthopaedics, Ninth edition, Volume one, Part VI, Chapter 17, incorporated by reference herein).

During use, expansion tool 7 is coupled to an inner edge 1, or inner edge region, of the implant 20 (in a manner described below) and, once at the implantation site, is rotated by a physician in order to unwind and expand the implant 20. This increases the implant's outer radius from r1 (FIG. 2) to r2 (FIG. 4) by about 5 mm to about 15 mm (e.g., about 7.5 mm). FIGS. 1-4 are not necessarily drawn to scale.

As appropriate, techniques described herein may be practiced using techniques and apparatus described in one or more of the references cited above in the Background section of the application.

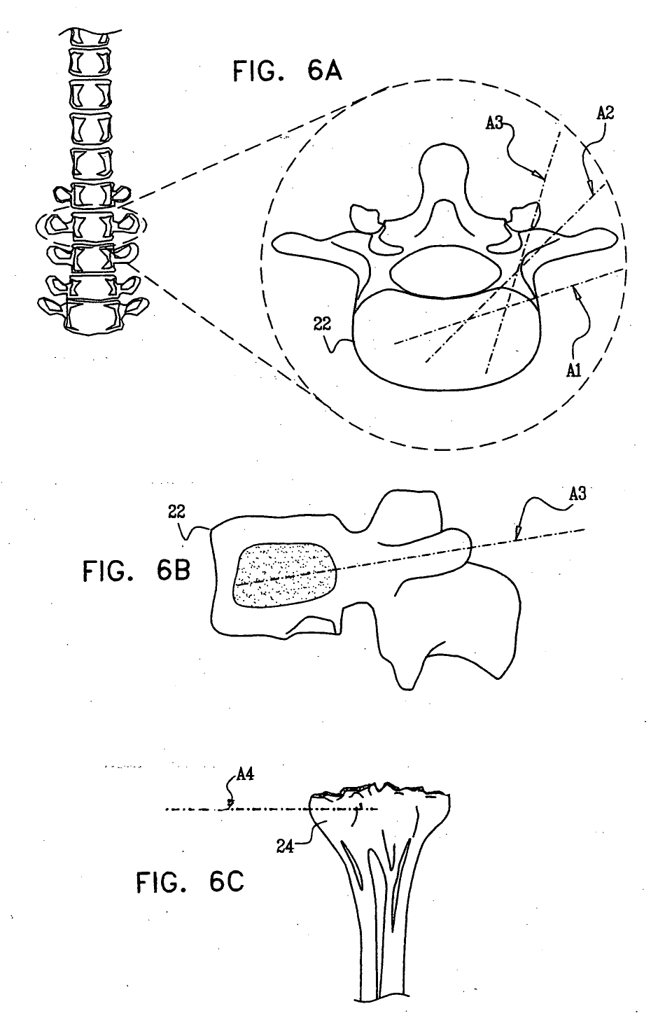

FIGS. 6A and 6B are schematic anatomical drawings showing suitable implantation approaches, in accordance with various embodiments of the present invention. Placement of implant 20 inside a vertebral body 22 is typically performed using one of three approaches: a lateral approach A1, a parapedicular approach A2, or a transpedicular approach A3. Using one or more drills of appropriate diameters, a hole is typically drilled in the outer portion of the vertebral body 22 in order to facilitate one of the these approaches. As appropriate, techniques cited hereinabove by Campbell for taking a biopsy, and/or in U.S. Patent Application Publication No. 2005/0038517, PCT Patent Publication Nos. WO 04/086934, WO 03/039328 and/or WO 04/034924 may be adapted for use in these embodiments.

FIG. 6C is a schematic anatomical drawing showing a suitable implantation approach for treating an upper tibial compression fracture, in accordance with an embodiment of the present invention. Placement of implant 20 inside a tibia 24 is typically performed using a lateral approach A4. It is to be understood that some embodiments of the present invention are described with respect to vertebral fractures by way of illustration and not limitation. The scope of the present invention includes application of these techniques in the treatment of other fractures, such as tibial fractures, as well, the implementation of which would be readily apparent to those skilled in the art.

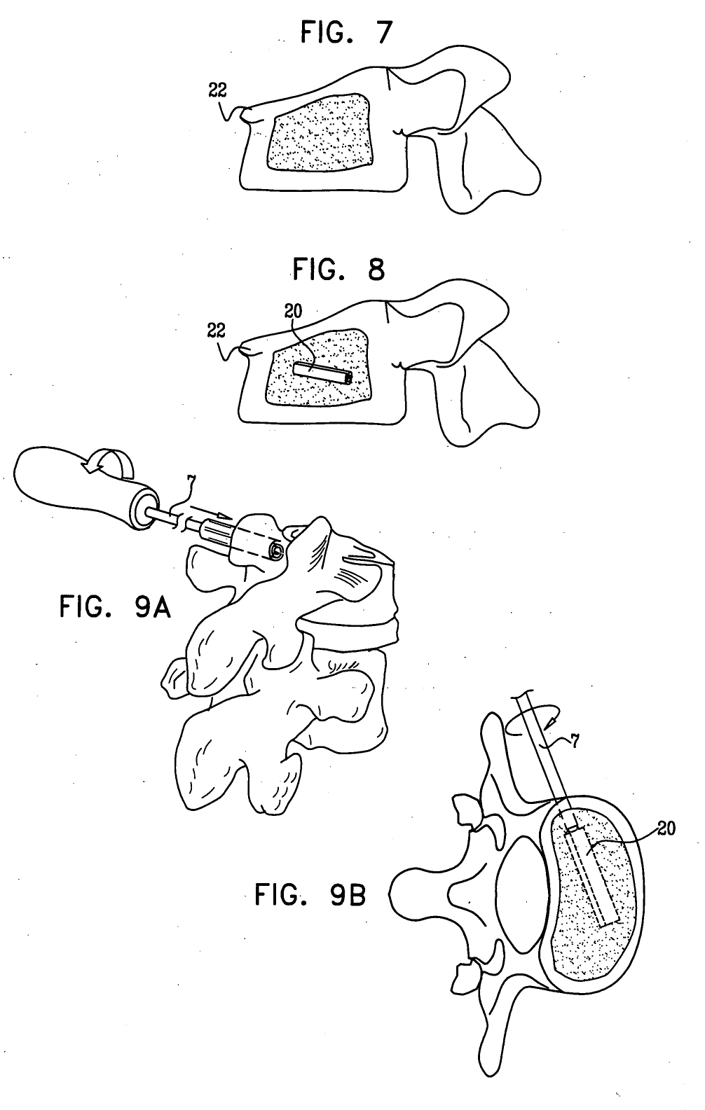

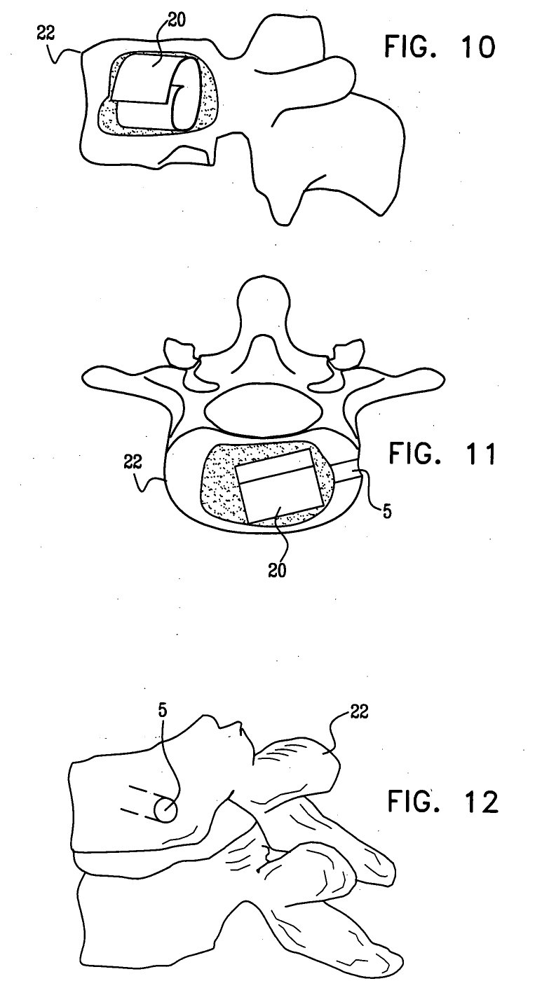

Reference is now made to FIGS. 7-12, which are schematic illustrations showing an implantation procedure, in accordance with an embodiment of the present invention. FIG. 7 shows a vertebral body 22 that has sustained a compression fracture. FIG. 8 shows the fractured vertebral body with implant 20 placed therein, still in the compressed state. FIG. 9 shows expansion tool 7 beginning to expand the implant inside the vertebral body (in this case, using approach A1 described above with reference to FIG. 6A). FIG. 10 shows implant 20 fully expanded inside vertebral body 22, after tool 7 has been withdrawn. FIG. 11 shows another perspective view of fully-expanded implant 20 inside the vertebral body, and a hole 5 in a portion of the vertebral body, through which the implant 20 was inserted. FIG. 12 shows vertebral body 22 after surgery, with its height generally restored by implant 20. (Implant 20 is not visible in FIG. 12.) Optionally, hole 5 is filled following the implantation and expansion of implant 20.

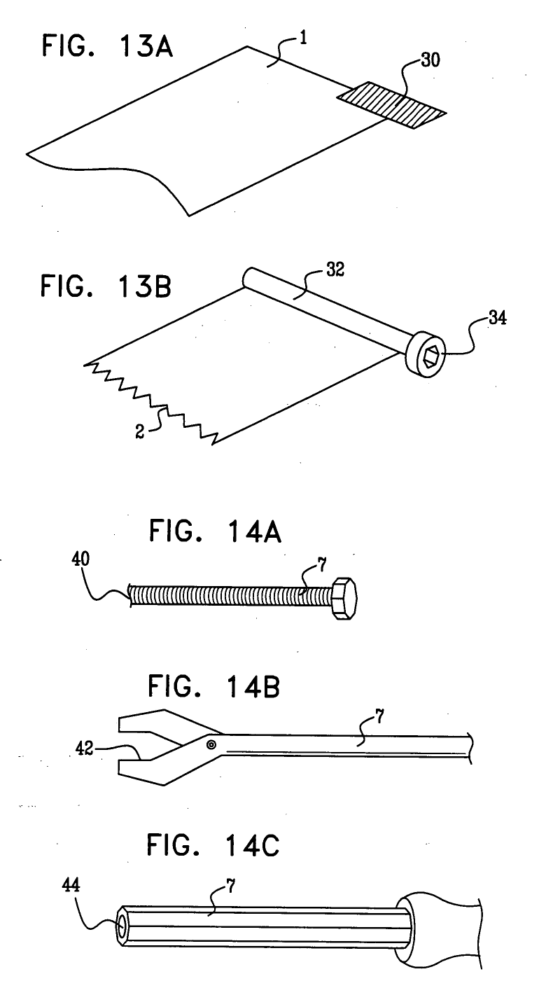

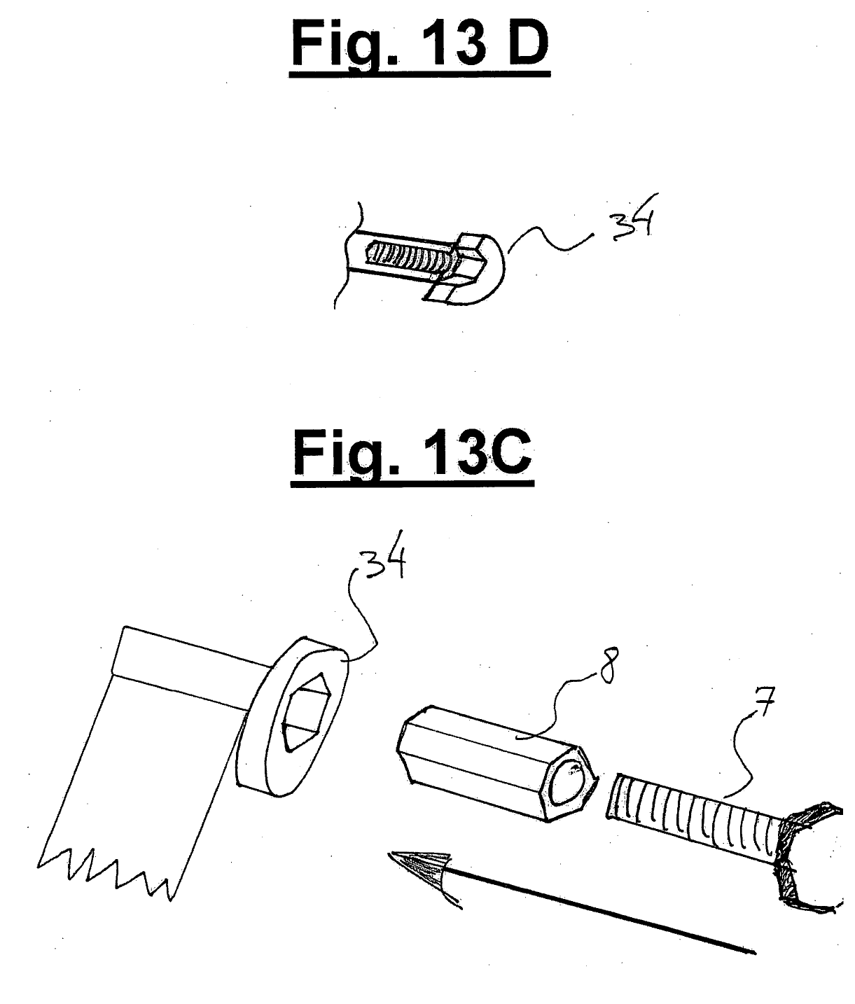

Referring now to FIGS. 13A, 13B, 13C and 13D, which are schematic illustrations of implant 20 in accordance with respective embodiments of the present invention, FIG. 13A shows implant 20 comprising a coupling member 30 on inner edge 1 thereof, or inner edge region, for coupling the implant 20 to expansion tool 7 (for example, enabling the tool 7 shown in FIG. 14B to removably grip coupling member 30 and enable rotation of the implant 20 upon rotation of expansion tool 7). Coupling member 30 may be a flat piece of material attached or otherwise adhered to the inner edge region of the implant 20.

Alternatively, a coupling member is provided which is shaped such that the expansion tool 7 couples thereto by means of any other available form of a coupling mechanism including but not limited to male-female type fittings known in the art (see FIG. 13C wherein a coupling portion 34 of coupling member 32 has a female fitting and a complementary male fitting or polygonal tip 8 is mounted onto a threaded expansion tool 7 or bolt) and threads (see FIG. 13D).

FIG. 13B shows a coupling member 32 of implant 20, which typically extends across the width of implant 20, but alternatively may extend across only a portion of the width of the implant 20, and comprises an elongate member, rod or shaft and a female coupling portion 34 by means of which expansion tool 7 sets the final, expanded state or size of implant 20. In the embodiment shown in FIG. 13B, an outer edge 2 of implant 20 is jagged, e.g., shaped to define teeth, and/or is shaped or otherwise treated (e.g., heat treated) to improve adhesion to vertebral body 22. Instead of teeth, other forms and constructions of the outer edge region of the implant 20 may be used to provide enhanced adhesion of the implant 20 to the bone.

As appropriate, coupling member 30, 32 or 34 extends partially across the width of implant 20, or completely across the width of implant 20.

For some applications, the outer 5 mm to 10 mm of implant 20 (i.e., the 5 mm to 10 mm closest to outer edge 2) is configured to enhance adhesion to vertebral body 22. With this enhanced adhesion, the physician is able to place implant 20 in the compressed state in the vertebral body and to firmly secure the implant 20 thereto. Once the outer edge 2 of the implant is firmly in place, rotation of expansion tool 7 is directly translated into a desired level of expansion of the implant 20. When teeth or other protruding members are used to provide the adhesion, they are typically less than 10 mm long, and/or otherwise configured, such that they do not penetrate the upper cortex bone of the vertebral body and/or damage the disc or other tissue outside of the vertebral body.

FIGS. 14A, 14B and 14C are schematic illustrations of distal tips of expansion tool 7, in accordance with respective embodiments of the present invention. The expansion tool 7 shown in FIG. 14A comprises a threaded tip 40, which engages a threaded portion of a coupling member such as coupling member 34 shown in FIG. 13D, or coupling member 34 shown in FIGS. 13B and 13C when engaged with polygonal tip 8 (shown in FIG. 13C). Coupling member 32 may be shaped to define a threaded portion within the longitudinal body thereof and/or within coupling portion 34.

FIG. 14B shows a grasping tip 42, which either grasps inner edge 1 of implant 20 directly, or grasps a portion of a coupling member 30 of the implant 20 (shown in FIG. 13A).

FIG. 14C shows a polygonal male tip 44 formed or otherwise arranged in connection with the expansion tool 7, which couples with a suitably shaped portion 34 of a coupling member 32 on implant 20 (e.g., as shown in FIGS. 13B and 13C). For some applications, the bore shown in tip 44 allows passage therethrough of threaded tip 40 (see FIG. 13C). A person of ordinary skill in the art having read the disclosure of the present patent application will realize that other coupling techniques are also suitable.

For some applications, a method is provided for treating a bone with a compression fracture. The method includes inserting a device in a compressed state into a first site of the bone. The device may comprise, as appropriate, a coil as described above, or other suitable components, such as a balloon, a spring, or forceps that can apply force directed outwardly. The device is expanded under physician control while the device is in the bone, to reduce a first location of the fracture. The device is thereafter compressed while at the first site, and moved within the bone to a second site, either without removing the device from the bone at all or removing the device from the bone and inserting it into the bone through a different access hole leading to the second site. The device is expanded under physician control while at the second site, to reduce a second location of the fracture. The device is typically moved to a plurality of locations within the bone in order to reduce the fracture at each of these locations, according to the nature of the fracture. In an embodiment, the device is maintained within the bone following the procedure, i.e., it remains implanted within the bone.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating a bone, comprising:
a coil having a compressed state and an expanded state, said coil being insertable into the bone when in its compressed state; and
an expansion tool arranged to be coupled to said coil when said coil is present in the bone and to enable manual expansion of said coil from said compressed state to said expanded state, said coil supporting the bone when in its expanded state in the bone.

2. The apparatus of claim 1, wherein said expansion tool comprises a portion arranged to engage an inner edge region of said coil and to enable manual expansion of said coil upon application of a rotational force to said inner edge region of said coil.

3. The apparatus of claim 1, wherein said coil comprises coupling means arranged at an inner edge region thereof for enabling said expansion tool to be coupled to said coil.

4. The apparatus of claim 3, wherein said coupling means comprises a flat piece of material arranged on said inner edge region of said coil, said expansion tool comprising a grasping tie for grasping said piece of material.

5. The apparatus of claim 3, wherein said coupling means comprise an elongate member having a first coupling portion at one end, said expansion tool having a second coupling portion arranged to matingly engage with said first coupling portion of said elongate member.

6. The apparatus of claim 4, wherein said first and second coupling portions comprise mating threads.

7. The apparatus of claim 4, wherein said first coupling portion is a polygon-shaped female coupling portion and said expansion tool comprises a corresponding polygonal male tip.

8. The apparatus of claim 1, wherein said coil includes at least one hole in a surface thereof.

9. The apparatus of claim 1, wherein said coil is structured and arranged to be manually returned to its compressed state while in the bone after attaining its expanded state to enable repositioning of said coil within the bone, and expansion following repositioning.

10. The apparatus of claim 1, wherein said coil is structured and arranged to be expandable when in the bone to a range of possible final levels of expansion whereby an actual final level of expansion thereof is determinable by use of said expansion tool.

11. The apparatus of claim 1, wherein said coil includes means arranged in connection with an outer edge for providing enhanced adhesion to the bone.

12. The apparatus of claim 11, wherein said means comprise teeth.

13. A method for treating a bone, comprising:
inserting a coil in a compressed state into the bone; and then
expanding the coil under manual control to cause the coil to attain an expanded state in which it supports the bone.

14. The method of claim 13, wherein the step of expanding the coil comprises engaging an expansion tool with the coil after insertion into the bone and then manipulating the expansion tool to expand the coil.

15. The method of claim 14, wherein the expansion tool is manipulated to set a final level of expansion of the coil.

16. The method of claim 13, wherein the bone includes a vertebral body, the step of inserting the coil comprising inserting the coil into the vertebral body.

17. The method of claim 13, wherein the bone includes a tibia, the step of inserting the coil comprising inserting the coil into the tibia.

18. The method of claim 13, wherein the step of expanding the coil comprises manually rotating an inner portion of the coil while an outer edge of the coil remains substantially stationary.

19. The method of claim 13, further comprising:
providing a plurality of holes in a surface of the coil; and
applying bone graft within the coil whereby the bone graft communicates through the holes with the bone.

20. The method of claim 13, further comprising:
compressing the coil in the bone after expanding the coil; then
repositioning the coil; and then
re-expanding the coil under manual control while the coil is in the bone.

21. The method of claim 13, further comprising:
compressing the coil in the bone after expanding the coil; and then
removing the compressed coil from the bone.

22. The method of claim 13, wherein the step of inserting the coil into the bone comprises securing an outer edge of the coil to native bone.

23. The method of claim 22, wherein the outer edge of the coil comprises teeth, the step of securing the outer edge of the coil to native bone comprising stabilizing the coil in the bone by coupling the teeth to bone tissue.

24. A method for treating a bone, comprising:
inserting a device in a compressed state into a first site in the bone;
expanding the device under manual control while the device is at the first site in the bone to reduce a first location of the fracture; then
compressing the device while the device is at the first site in the bone; then
moving the device to a second site in the bone; and then
expanding the device under manual control while the device is at the second site in the bone to reduce a second location of the fracture.

25. The method of claim 24, wherein the step of inserting the device comprises inserting the device during a medical procedure, further comprising maintaining the device within the bone following the procedure.
